(19) United States
(12) Patent Application Publication
(10) Pub. No.: US 2023/0154599 A1
(43) Pub. Date: May 18, 2023

(54) SYSTEM AND METHOD FOR IDENTIFYING OPTIMAL APPOINTMENT TIMES OF PATIENTS

(71) Applicant: Smirta Innovations Inc., Holmdel, NJ (US)

(72) Inventors: Shridar Iyengar, Morganville, NJ (US); Paramasiviah Harshavardhana, Marlboro, NJ (US); Ram Iyengar, Holmdel, NJ (US); Bhaskar Anepu, Allentown, PA (US)

(21) Appl. No.: 18/054,620

(22) Filed: Nov. 11, 2022

Related U.S. Application Data
(60) Provisional application No. 63/279,086, filed on Nov. 13, 2021.
(60) Provisional application No. 63/297,258, filed on Jan. 7, 2022.

(51) Int. Cl. G16H 40/20; G16H 10/60; G06Q 10/10

(52) U.S. Cl. CPC G16H 40/20; G16H 10/60; G06Q 10/1095

(57) ABSTRACT

A system and method for identifying optimal appointment times of patients is disclosed. The method includes receiving a request from one or more electronic devices associated with a scheduler, receiving one or more inputs associated with the patient from an ERR system based on the received request, and obtaining a clinic configuration input from a statistically optimized DOW template. Further, the method includes determining one or more time slots for the patient profile of the patient, generating an optimal rank corresponding to each of the determined one or more time slots, and generating one or more recommendations for each of the determined one or more time slots. Furthermore, the method includes outputting the one or more time slots for the treatment date in accordance with their optimal rank and the one or more recommendations on user interface screen of the one or more electronic devices associated with the scheduler.

### CROSS-REFERENCE TO RELATED APPLICATIONS

This Application claims priority from a patent application filed in the US having Patent Application No. 63/279,086 filed on Nov. 13, 2021 and titled “METHODS TO PROVIDE OPTIMAL, ADAPTIVE, AND REAL-TIME PATIENT SCHEDULING AT CANCER TREATMENT FACILITIES”. It also claims priority from a patent application filed in the U.S. patent application Ser. No. 63/297,258 filed on Jan. 7, 2022 and titled “SYSTEM AND METHOD FOR CREATING OPTIMIZED DAY OF WEEK TEMPLATE BASED ON HISTORIC PATIENT DATA”

### FIELD OF INVENTION

Embodiments of the present disclosure relate to patient treatment systems, and more particularly relates to a system and method for identifying optimal appointment times of patients.

### BACKGROUND

Patient scheduling is a process of assigning individual patients and/or patients' activities to a specific time and/or healthcare resources. Generally, patient scheduling at healthcare facilities is extremely complex. The volume of patients on any specific day in the future is highly variable. There is also the impact of cancellations, add-ons, and no-shows, and the mix of treatment durations for a given day. This becomes a central issue of treatment scheduling that creates a challenge for schedulers. It creates a logistical challenge that is beyond the capacity of a normal human mind to solve, specifically in a short amount of time with limited information that is available at the time of scheduling a patient. Further, sub-optimal scheduling tends to result in long patient wait times, imbalanced treatment chair utilization across a given day, and uneven nurse load resulting in high stress levels.

For example, cancer treatment schedules are unique and may differ significantly from one day of a week (DOW) to another DOW. Standard scheduling solutions do not offer any insight into patient flow patterns or resource utilization distribution. Optimized scheduling of patients at cancer treatment facilities require consideration of several moving parts. Performing this task without an understanding of how different treatment profiles are distributed for any given DOW is not possible. The result is an imbalance in patient flow and resource utilization. Combinatorial optimization, however, is challenging on account of a sheer number of combinatorial options involved. A computational complexity entailed in determining a best mix of options is prohibitive.

Hence, there is a need for an improved system and method for identifying optimal appointment times of patients, in order to address the aforementioned issues.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

In accordance with an embodiment of the present disclosure, a computing system for identifying optimal appointment times of patients is disclosed. The computing system includes one or more hardware processors and a memory coupled to the one or more hardware processors. The memory includes a plurality of modules in the form of programmable instructions executable by the one or more hardware processors. The plurality of modules include a request receiver module configured to receive a request from one or more electronic devices associated with a scheduler to determine an optimal appointment time for a patient. The request includes a patient ID of the patient, a patient profile of the patient and a treatment date. The patient profile of the patient includes one or more medical services required by the patient. The plurality of modules also include an input receiver module configured to receive one or more inputs associated with the patient from an Electronic Health Record (EHR) system based on the received request. The one or more inputs include a patient Medical Record Number (MRN), the treatment date, a treatment schedule location, one or more required resources, availability of the one or more required resources, a resource duration, schedule of staff, a number of patients assigned to each time slot, and one or more services required by each of the patients. Furthermore, the plurality of modules also include a data obtaining module configured to obtain a clinic configuration input from a statistically optimized Day of the Week (DOW) template. The statistically optimized DOW template includes forecasted patient profiles assigned to optimized time slots. Furthermore, the plurality of modules include a schedule determination module configured to determine one or more time slots for the patient profile of the patient based on the received one or more inputs and the obtained clinic configuration input. The one or more time slots are determined for the treatment date and a predefined clinic. The plurality of modules include a rank generation module configured to generate an optimal rank corresponding to each of the determined one or more time slots based on the received one or more inputs, the obtained clinic configuration input, and one or more patient preferences. A time slot with highest optimal rank is a most optimal time slot among the generated one or more time slots for the patient. Further, a time slot with lowest optimal rank is a least optimal time slot among the generated one or more time slots for the patient. The plurality of modules include a recommendation generation module configured to generate one or more recommendations for each of the determined one or more time slots based on the received one or more inputs, the obtained clinic configuration input, and one or more patient preferences upon generating the optimal rank. Furthermore, the plurality of modules include a data output module configured to output the determined one or more time slots for the treatment date in accordance with their optimal rank and the generated one or more recommendations on user interface screen of the one or more electronic devices associated with the scheduler.

In accordance with another embodiment of the present disclosure, a method identifying optimal appointment times of patients is disclosed. The method includes receiving a request from one or more electronic devices associated with a scheduler to determine an optimal appointment time for a patient. The request includes a patient ID of the patient, a patient profile of the patient and a treatment date. The patient profile of the patient includes one or more medical services required by the patient. The method further includes receiving one or more inputs associated with the patient from an Electronic Health Record (EHR) system based on the received request. The one or more inputs include a patient Medical Record Number (MRN), the treatment date, a treatment schedule location, one or more required resources, availability of the one or more required resources, a resource duration, schedule of staff, a number of patients assigned to each time slot, and one or more services required by each of the patients. Further, the method includes obtaining a clinic configuration input from a statistically optimized Day of the Week (DOW) template. The statistically optimized DOW template includes forecasted patient profiles assigned to optimized time slots. Furthermore, the method includes determining one or more time slots for the patient profile of the patient based on the received one or more inputs and the obtained clinic configuration input. The one or more time slots are determined for the treatment date and a predefined clinic. The method includes generating an optimal rank corresponding to each of the determined one or more time slots based on the received one or more inputs, the obtained clinic configuration input, and one or more patient preferences. A time slot with highest optimal rank is a most optimal time slot among the generated one or more time slots for the patient. Further a time slot with lowest optimal rank is a least optimal time slot among the generated one or more time slots for the patient. The method includes generating one or more recommendations for each of the determined one or more time slots based on the received one or more inputs, the obtained clinic configuration input, and one or more patient preferences upon generating the optimal rank. Furthermore, the method includes outputting the determined one or more time slots for the treatment date in accordance with their optimal rank and the generated one or more recommendations on user interface screen of the one or more electronic devices associated with the scheduler.

Embodiment of the present disclosure also provide a non-transitory computer-readable storage medium having instructions stored therein that, when executed by a hardware processor, cause the processor to perform method steps as described above.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF DRAWINGS

The disclosure will be described and explained with additional specificity and detail with the accompanying figures in which:

FIG. 1 is a block diagram illustrating an exemplary computing environment for identifying optimal appointment times of patients, in accordance with an embodiment of the present disclosure;

FIG. 2 is a block diagram illustrating an exemplary computing system for identifying optimal appointment times of patients, in accordance with an embodiment of the present disclosure;

FIG. 3 is a block diagram illustrating an exemplary operation of the computing system for identifying optimal appointment times of patients, in accordance with an embodiment of the present disclosure;

FIG. 4 is a graphical user interface screen of the computing system for identifying optimal appointment times of patients, in accordance with an embodiment of the present disclosure;

FIG. 5 is a block diagram depicting key steps involved in generation of optimized Day of the Week (DOW) template, in accordance with an embodiment of the present disclosure;

FIG. 6 is a tabular representation depicting an illustrative forecasted profile for a specific DOW, in accordance with an embodiment of the present disclosure;

FIG. 7 is a block diagram depicting key steps within optimization module for determining DOW template, in accordance with an embodiment of the present disclosure;

FIG. 8 is a tabular representation depicting an illustrative optimized DOW template, in accordance with an embodiment of the present disclosure; and

FIG. 9 is a process flow diagram illustrating an exemplary method for identifying optimal appointment times of patients, in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE DISCLOSURE

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure. It will be understood by those skilled in the art that the foregoing general description and the following detailed description are exemplary and explanatory of the disclosure and are not intended to be restrictive thereof.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprise”, “comprising”, or any other variations thereof, are intended to cover a non-exclusive inclusion, such that one or more devices or sub-systems or elements or structures or components preceded by “comprises . . . a” does not, without more constraints, preclude the existence of other devices, sub-systems, additional sub-modules. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

A computer system (standalone, client or server computer system) configured by an application may constitute a “module” (or “subsystem”) that is configured and operated to perform certain operations. In one embodiment, the “module” or “subsystem” may be implemented mechanically or electronically, so a module include dedicated circuitry or logic that is permanently configured (within a special-purpose processor) to perform certain operations. In another embodiment, a “module” or “subsystem” may also comprise programmable logic or circuitry (as encompassed within a general-purpose processor or other programmable processor) that is temporarily configured by software to perform certain operations.

Accordingly, the term “module” or “subsystem” should be understood to encompass a tangible entity, be that an entity that is physically constructed permanently configured (hardwired) or temporarily configured (programmed) to operate in a certain manner and/or to perform certain operations described herein.

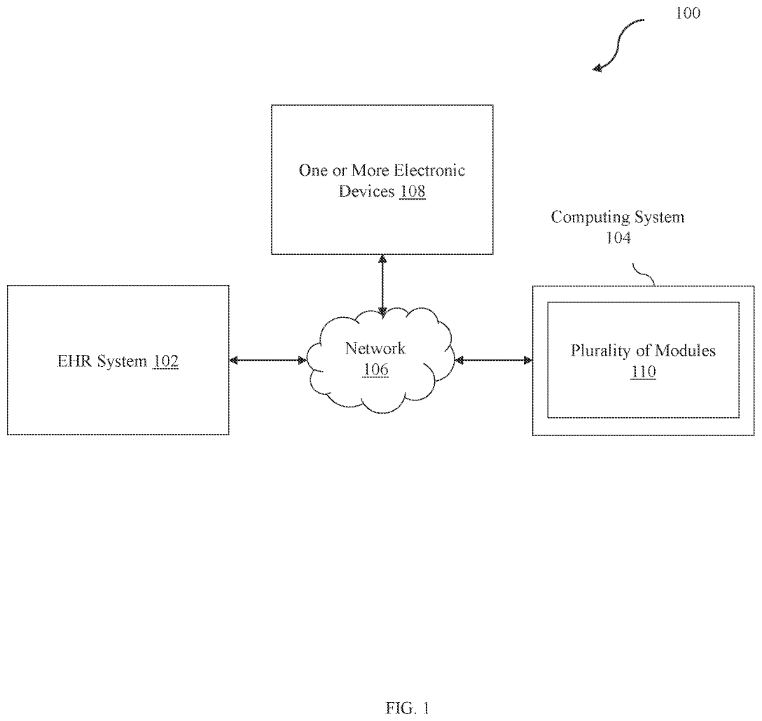

Referring now to the drawings, and more particularly to FIGS. 1 through FIG. 9, where similar reference characters denote corresponding features consistently throughout the figures, there are shown preferred embodiments and these embodiments are described in the context of the following exemplary system and/or method.

FIG. 1 is a block diagram illustrating an exemplary computing environment 100 for identifying optimal appointment times of patients, in accordance with an embodiment of the present disclosure. According to FIG. 1, the computing environment 100 includes an Electronic Health Record (EHR) system 102 communicatively coupled to a computing system 104 via a network 106. In an embodiment of the present disclosure, the EHR system 102 is an external database one or more inputs. Further, the network 106 may be internet or any other wireless network. The computing system 104 may be hosted on a central server, such as cloud server or a remote server.

Further, the computing environment 100 includes one or more electronic devices 108 associated with a scheduler communicatively coupled to the computing system 104 via the network 106. In an embodiment of the present disclosure, the scheduler is a user who schedules appointment of one or more patients at a healthcare facility. In an exemplary embodiment of the present disclosure, the healthcare facility includes ambulatory surgical centres, blood banks, clinics and medical offices, dialysis centres, hospice homes, hospitals, imaging and radiology centres, and the like. In an embodiment of the present disclosure, the one or more electronic devices 108 are configured to receive the request from the scheduler to determine an optimal appointment time for the patient. The one or more electronic devices 108 also output one or more time slots for the treatment date in accordance with their optimal rank and one or more recommendations to the computing system 104. In an exemplary embodiment of the present disclosure, the one or more electronic devices 108 may include a laptop computer, desktop computer, tablet computer, smartphone, wearable device, a digital camera and the like.

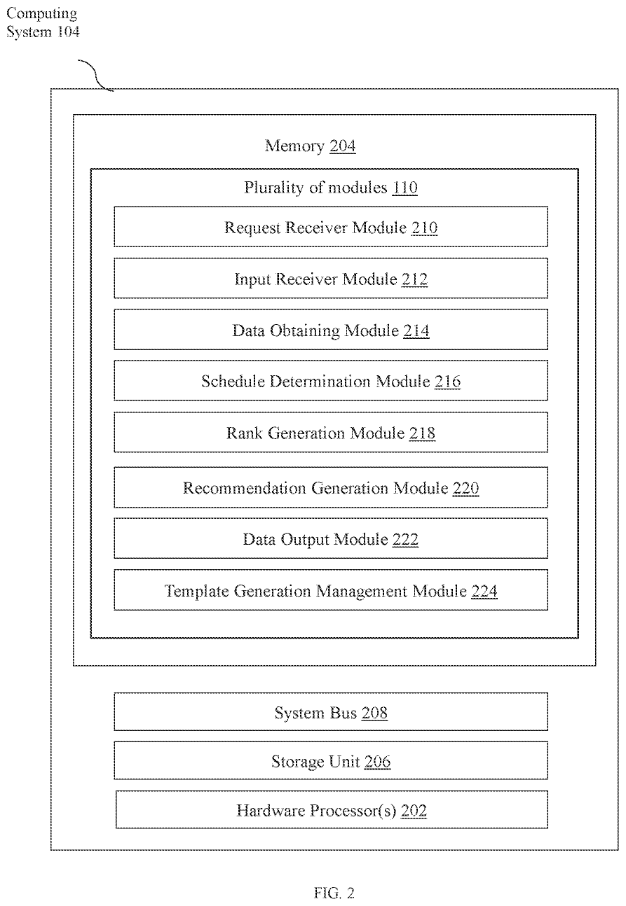

Furthermore, the one or more electronic devices 108 include a local browser, a mobile application or a combination thereof. Furthermore, the scheduler may use a web application via the local browser, the mobile application or a combination thereof to communicate with the computing system 104. In an exemplary embodiment of the present disclosure, the mobile application may be compatible with any mobile operating system, such as android, iOS, and the like. In an embodiment of the present disclosure, the computing system 104 includes a plurality of modules 110. Details on the plurality of modules 110 have been elaborated in subsequent paragraphs of the present description with reference to FIG. 2.

In an embodiment of the present disclosure, the computing system 104 is configured to receive a request from the one or more electronic devices 108 associated with the scheduler to determine an optimal appointment time for a patient. Further, the computing system 104 receives one or more inputs associated with the patient from the EHR system 102 based on the received request. Furthermore, the computing system 104 obtains a clinic configuration input from a statistically optimized DOW template. The computing system 104 determines one or more time slots for the patient profile of the patient based on the received one or more inputs and the obtained clinic configuration input. Further, the computing system 104 generates an optimal rank corresponding to each of the determined one or more time slots based on the received one or more inputs, the obtained clinic configuration input, and one or more patient preferences. The computing system 104 generates one or more recommendations for each of the determined one or more time slots based on the received one or more inputs, the obtained clinic configuration input, and one or more patient preferences upon generating the optimal rank. Further, the computing system 104 outputs the determined one or more time slots for the treatment date in accordance with their optimal rank and the generated one or more recommendations on user interface screen of the one or more electronic devices 108 associated with the scheduler.

FIG. 2 is a block diagram illustrating an exemplary computing system 104 for identifying optimal appointment times of patients, in accordance with an embodiment of the present disclosure. Further, the computing system 104 includes one or more hardware processors 202, a memory 204 and a storage unit 206. The one or more hardware processors 202, the memory 204 and the storage unit 206 are communicatively coupled through a system bus 208 or any similar mechanism. The memory 204 comprises the plurality of modules 110 in the form of programmable instructions executable by the one or more hardware processors 202. Further, the plurality of modules 110 includes a request receiver module 210, an input receiver module 212, a data obtaining module 214, a schedule determination module 216, a rank generation module 218, a recommendation generation module 220, a data output module 222, and a template generation management module 224.

The one or more hardware processors 202, as used herein, means any type of computational circuit, such as, but not limited to, a microprocessor unit, microcontroller, complex instruction set computing microprocessor unit, reduced instruction set computing microprocessor unit, very long instruction word microprocessor unit, explicitly parallel instruction computing microprocessor unit, graphics processing unit, digital signal processing unit, or any other type of processing circuit. The one or more hardware processors 202 may also include embedded controllers, such as generic or programmable logic devices or arrays, application specific integrated circuits, single-chip computers, and the like.

The memory 204 may be non-transitory volatile memory and non-volatile memory. The memory 204 may be coupled for communication with the one or more hardware processors 202, such as being a computer-readable storage medium. The one or more hardware processors 202 may execute machine-readable instructions and/or source code stored in the memory 204. A variety of machine-readable instructions may be stored in and accessed from the memory 204. The memory 204 may include any suitable elements for storing data and machine-readable instructions, such as read only memory, random access memory, erasable programmable read only memory, electrically erasable programmable read only memory, a hard drive, a removable media drive for handling compact disks, digital video disks, diskettes, magnetic tape cartridges, memory cards, and the like. In the present embodiment, the memory 204 includes the plurality of modules 110 stored in the form of machine-readable instructions on any of the above-mentioned storage media and may be in communication with and executed by the one or more hardware processors 202.

In an embodiment of the present disclosure, the storage unit 206 may be a cloud storage. The storage unit 206 may store the received request, the one or more inputs, the clinic configuration input, the one or more time slots, the optimal rank, the one or more recommendations, one or more required resources, one or more patient preferences, one or more approximate matches, a set of static optimized Day of the Week (DOW) templates and the like.

The request receiver module 210 is configured to receive the request from the one or more electronic devices 108 associated with the scheduler to determine an optimal appointment time for a patient. In an exemplary embodiment of the present disclosure, a patient ID of the patient, a patient profile of the patient, a treatment date, and the like. In an embodiment of the present disclosure, the patient profile of the patient includes one or more medical services required by the patient. In an exemplary embodiment of the present disclosure, the patient profile corresponds to any combination of different medical services such as, but not limited to, lab tests, appointment with Medical Assistant (MA), appointment with a physician or a nurse practitioner (MD), injection, treatment, and the like. For example, the one or more medical services include injection, treatment, lab tests required to be performed on the patient, and the like. In an exemplary embodiment of the present disclosure, the one or more electronic devices 108 may include a laptop computer, desktop computer, tablet computer, smartphone, wearable device, a digital camera and the like.

The input receiver module 212 is configured to receive one or more inputs associated with the patient from the EHR system 102 based on the received request. In an exemplary embodiment of the present disclosure, the one or more inputs include a patient Medical Record Number (MRN), the treatment date, a treatment schedule location, one or more required resources, availability of the one or more required resources, a resource duration, schedule of staff, a number of patients assigned to each time slot, one or more services required by each of the patients, and the like. For example, the one or more required resources include lab chair, treatment chair, hospital sketcher, defibrillators used as part of patient treatment procedure, blood glucose meters, Magnetic Resonance Imaging (MRI), or any combination thereof. In an exemplary embodiment of the present disclosure, the staff includes availability of medical assistant, nurse, doctor, clinician, and the like.

The data obtaining module 214 is configured to obtain a clinic configuration input. In an exemplary embodiment of the present disclosure, the clinic configuration input includes, but not limited to, clinic operating hours, staff availability first treatment start time, last treatment end time and the like. For example, first lab starts at 7 am and last lab ends at 3 pm, resource counts such as number of treatment chairs, number of injections chairs, number of lab chairs, staff counts and availability constraints. For instance, any treatments should not begin between 12 PM and 1 PM, 80% of treatments less than 90 mins must be scheduled after 1 PM and the like.

The schedule determination module 216 is configured to determine the one or more time slots for the patient profile of the patient based on the received one or more inputs and the obtained clinic configuration input. In an embodiment of the present disclosure, the one or more time slots are determined for the treatment date and a predefined clinic. The method to determine one or more slots includes the following steps, firstly, all the services included in the patient visit request are considered. For example, 15 min Lab followed by a 15 min MD visit followed by a 90 mm treatment. Secondly, Staff availability and clinic constraints for the day of the visit is considered. For example, if the lab is available from 8 AM to 3 PM and the MD is available between 9 AM and 2 PM. Treatments less than or equal to 90 mins cannot be scheduled before 1 PM. Lastly, based on the aforementioned request in the first step and configuration details in the second step, optimal time slots for the visit are configured as follows: 12:30 PM lab followed by 12:45 PM MD visit followed by 1:00 PM treatment, 12:45 PM lab followed by 1:00 PM MD visit followed by 1:15 PM treatment, 1:00 PM lab followed by 1:15 PM MD visit followed by 1:30 PM treatment.

The rank generation module 218 is configured to generate the optimal rank corresponding to each of the determined one or more time slots based on the received one or more inputs, the obtained clinic configuration input, and one or more patient preferences. In an embodiment of the present disclosure, a time slot with highest optimal rank is a most optimal time slot among the generated one or more time slots for the patient. In an embodiment of the present disclosure, a time slot with lowest optimal rank is a least optimal time slot among the generated one or more time slots for the patient. For example, the one or more patient preferences include a preferred time, a preferred staff a preferred location of a clinic, a preferred treatment date, and the like. In generating the optimal rank corresponding to each of the determined one or more time slots based on the received one or more inputs, the obtained template input, and the one or more patient preferences, the rank generation module 218 correlates the determined one or more time slots, the received one or more inputs, the obtained template input, and the one or more patient preferences. Further, the rank generation module 218 generates the optimal rank corresponding to each of the determined one or more time slots based on result of correlation. For example, the computing system 104 takes into consideration different resources required by the specific treatment profile and recommends rank ordered time slots based on the EHR data and the static optimization template. The computing system 104 achieves this by combining information pertaining to the time slots assigned to various treatment profiles in the static DOW template with the specifics of the EHR data for the chosen clinic for the chosen day.

The method used by the rank generation module 218 for an optimal rank generation in order to display the time slots follows a sequence of steps. The optimal rank generation is based on a visit request matching optimal times in the static DOW template. In an exemplary embodiment of the present disclosure, if 15 min MD visit followed by 120 min treatment is requested, the following steps are considered for optimal rank generation, for the optimal times available in the static DOW template. In the first step, 9:00 AM MD visit and 9:15 AM 120 min treatment is assigned rank 1, since the aforementioned schedule is an exact match. In the second step, 12:00 PM MD visit and 12:15 PM 150 min treatment is assigned rank 2. Since the aforementioned schedule is not an exact match. As 150 min treatment exceeds 30 mins when compared to the requested 120 min slot. In the third step, 10:00 AM MD visit and 10:15 AM 180 min treatment are assigned rank 3. Since the aforementioned schedule is not an exact match as 180 mins is 60 mins more than the requested 120 mins slot.

The recommendation generation module 220 is configured to generate the one or more recommendations for each of the determined one or more time slots based on the received one or more inputs, the obtained clinic configuration input, and one or more patient preferences upon generating the optimal rank. In an exemplary embodiment of the present disclosure, the one or more recommendations include waiting time recommendations, staff availability recommendations, resource recommendations, treatment date recommendations, and the like. For example, the one or more recommendations may be that ‘the patient may visit a clinic at the preferred time, but he may have to wait for 15 minutes.’ In another example, the one or more recommendations may be that ‘the patient may visit a clinic at the preferred time, but the one or more medical services may not be delivered by the preferred staff.’

The data output module 222 is configured to output the determined one or more time slots for the treatment date in accordance with their optimal rank and the generated one or more recommendations on user interface screen of the one or more electronic devices 108 associated with the scheduler.

In an embodiment of the present disclosure, the template generation management module 224 receives historical patient data associated with patients from the EHR system 102. In an exemplary embodiment of the present disclosure, the historical patient data include service date, unique patient identifier, a breakdown of different services required for each visit, staff schedules, operating hours of a provider, and the like. For example, the provider may be, but not limited to a physician, group of physicians, clinic, facility that is part of a hospital or a health system, or any other person(s) or an entity that provides treatment to patients. Further, the template generation management module 224 generates the set of statistically optimized DOW templates for each DOW based on the received historic patient data and one or more constraints by using a statistical and combinatorial optimization analysis technique. In an embodiment of the present disclosure, the set of statistically optimized DOW templates include forecasted patient profiles for each DOW. In an exemplary embodiment of the present disclosure, the set of statistically optimized DOW templates correspond to a complete schedule for each forecasted patient profile. For example, the one or more constraints include provider schedules, staff availability, resource availability and clinic operating hours.

In another embodiment of the present disclosure, the forecasted patient profiles for each DOW correspond to the projected patient visits for future days based on historical data. For example, On Mondays, 50 patients come for 15 min lab and 15 min MD visit, 20 patients come for 15 min MD visit and 60 min treatment, 25 patients come for 15 min MD visit and 120 min treatment and the like. These time slots are deduced based on standard predictive modelling algorithms. Further, the set of statistically optimized DOW templates correspond to a representation of different starting times for different patient visits as described in the aforementioned example, in a way that the clinic resources are utilized efficiently. Visual representation of a DOW template is as illustrated in FIG. 8. Furthermore, the set of statistically optimized DOW templates are generated using statistical and predictive modelling techniques.

In a use-case scenario, a treatment profile may include one or more services. The static DOW template for this treatment profile may give many possible start times of T1, T2, . . . , TN. This recommended set of start times in conjunction with information obtained from the EHR may result in prioritizing the time slots T1, T2 . . . TN into an ordered list Ti1, Ti2, . . . TiN This prioritized list is then presented to the end user as part of a computer program. For example, a treatment profile (Tx1) may consist of 15 minutes lab, and 120 minutes treatment. This treatment profile, Tx1, may need to be scheduled for a date in the future that may be a Wednesday. Based on the static DOW template for a Wednesday, there may be three, time options when Tx1 may start; 10:00 AM (T1), 10:30 AM (T2), and 1:00 PM (T3). Information obtained from the ERR may indicate sufficient resource availability for all three start times T1, T2, and T3. Based on other considerations such as nurse availability, T2 may be considered more optimal than T3 and T3 more optimal than T1. Hence, the start times for Tx1 presented to the scheduler may be T2, T3, and T1 in that order.

In another use-case scenario, a treatment profile may include one or more services. The static day of the week template for the schedule date corresponding to this treatment profile may include an ordered list of times corresponding to “exact matches” and “approximate matches”. The “exact matches” are those that correspond exactly to the specific treatment profile under consideration whereas “approximate matches” are those that correspond in an approximate manner to the specific treatment profile under consideration. For example, a treatment profile may consist of two services, 15 minutes Lab and 60 minutes Treatment. Examples of “exact matches” for this treatment profile are: 08:00 15 minutes Lab, 08:15 60 minutes Treatment, 10:30 15 minutes Lab, 10:45 60 minutes Treatment and 15:00 15 minutes Lab, 15:15 60 minutes Treatment. Examples of “approximate matches” are: 09:30 15 minutes Lab, 09:45 90 minutes Treatment, 11:00 15 minutes Lab, 11:30 60 minutes Treatment, 14:00 30 minutes Lab, 14:15 60 minutes Treatment.

In another use-case scenario, the preferred embodiment may allow the scheduler to manually select any desired time slot or time slots for a specific treatment profile. The manual option may be used by the scheduler either when no “exact matches” or “approximate matches” are available, or when the scheduler wishes to make a selection other than the recommended optimized time slot or time slots.

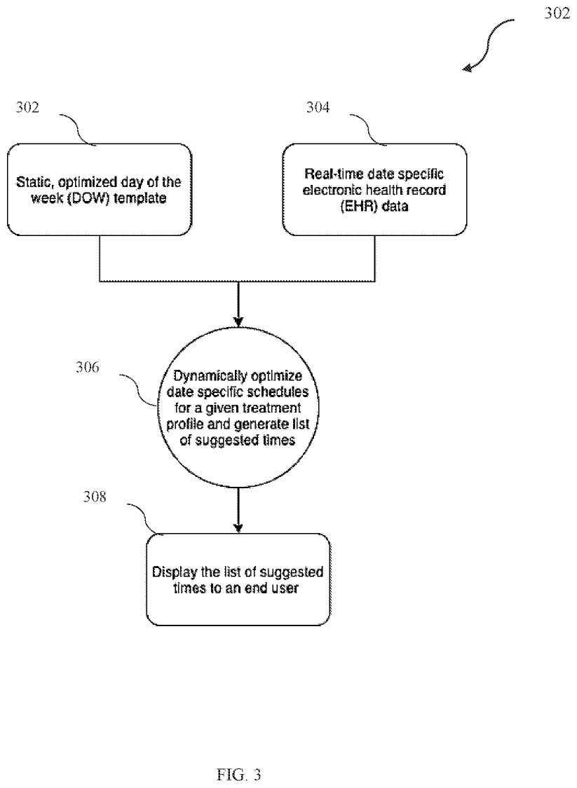

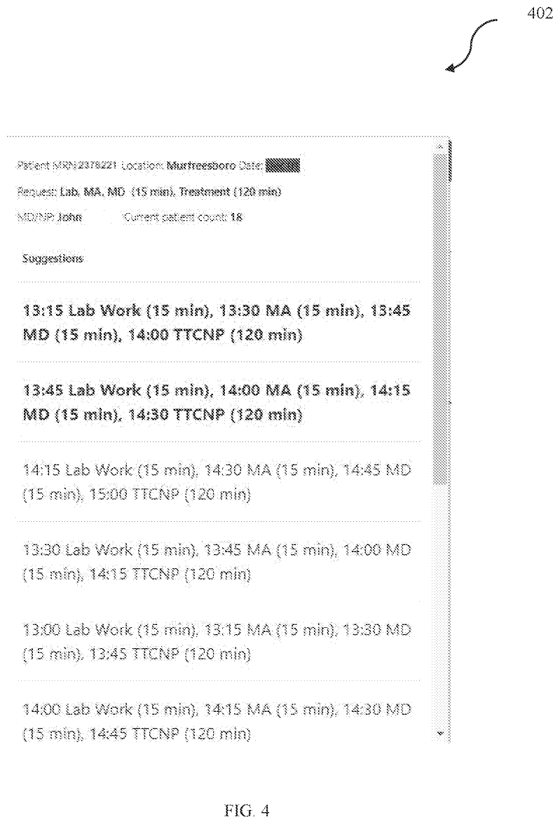

FIG. 3 is a block diagram illustrating an exemplary operation of the computing system 104 for identifying optimal appointment times of patients, in accordance with an embodiment of the present disclosure. The static and optimized DOW template 302 and the real-time date specific EHR data 304 are received by the computing system 104. Further, at step 306, data specific schedules are dynamically optimized for a given treatment profile and generate list of suggested times. At step 308, display the list of suggested times to an end user.

FIG. 4 is a graphical user interface screen of the computing system 104 for identifying optimal appointment times of patients, in accordance with an embodiment of the present disclosure. The graphical user interface screen 402 of FIG. 4 depicts an example where the optimized times suggested by the computing system 104 are presented to the end user as part of a computer software program. In this example, exact matches between the user requested treatment profile and DOW template suggested treatment profile are highlighted in bold and prioritized along with approximate matches.

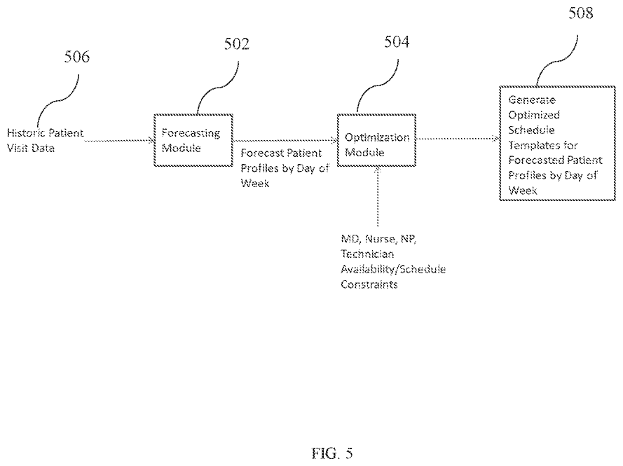

FIG. 5 is a block diagram depicting key steps involved in generation of optimized DOW template, in accordance with an embodiment of the present disclosure. The present computing system 104 includes a forecasting module 502 and an optimization module 504. Multiple optimized DOW templates are generated from historic patient data 506 employing a variety of statistical and combinatorial optimization analysis with the help of the optimization module 504. The statistical forecasting techniques utilized are common in the industry and familiar to the practitioners of the art. At step 508, a result of forecasting analysis from the forecasting module 502 is a set of DOW profiles, one for each DOW.

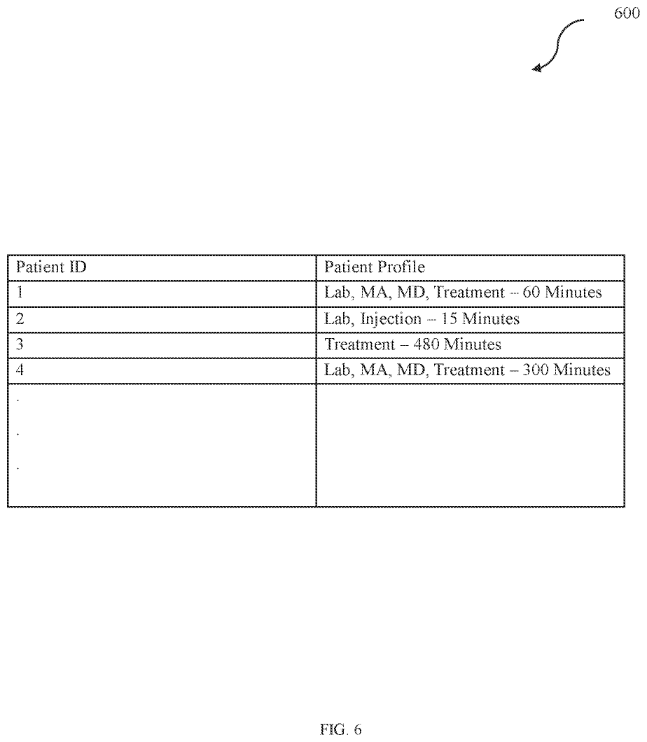

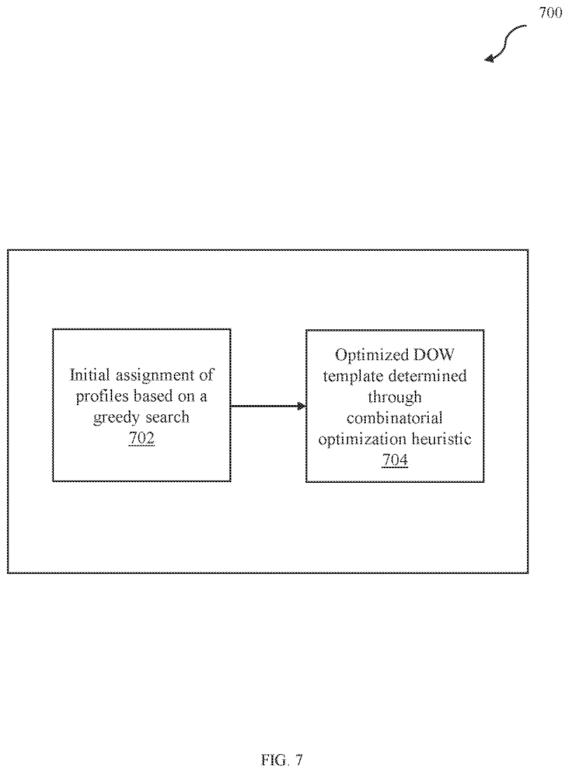

FIG. 6 is a tabular representation 600 depicting an illustrative forecasted profile for a specific DOW, in accordance with an embodiment of the present disclosure. The illustrative forecasted profile for the specific DOW includes various patient ID's and patient profiles. In one of the cases, the patient profile with a patient ID one has a lab, requires MA, has an appointment with physician or nurse practitioner (MD) and requires treatment for sixty minutes. Further, in another case, the patient profile with a patient ID two has the lab and requires application of injection for fifteen minutes. Further, the patient profile with a patient ID three requires treatment for four hundred eighty minutes. Further, the patient profile with a patient ID four has lab, requires MA, has an appointment with physician or nurse practitioner (MD) and requires treatment for three hundred minutes.

In an embodiment, the computing system 104 employs a novel optimization heuristic whereby an approximate solution is first found using a search approach commonly known to practitioners of the art. An intelligent optimization heuristic considers an approximate solution as starting point and generates most optimized solution taking various constraints into account such as, but not limited to, provider schedules, staff availability, resource availability and clinic operating hours to generate a complete schedule for each forecasted treatment profile. The express objective of an optimization process is to balance a load on a staff while ensuring that all resources are optimally used and number of treatment profiles that can be accommodated is maximized.

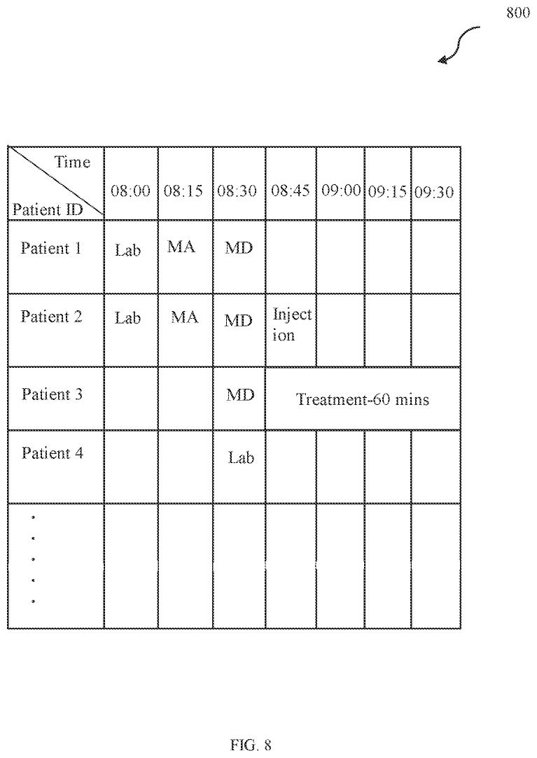

FIG. 7 is a block diagram 700 depicting key steps within optimization module for determining DOW template, in accordance with an embodiment of the present disclosure. A net result of this optimization module 504 is a DOW template for each DOW. At step 702, an initial assignment of profiles is performed based on a search. At step 704, a DOW template is optimized through combinational optimization heuristic.

FIG. 8 is a tabular representation 800 depicting an illustrative optimized DOW template, in accordance with an embodiment of the present disclosure. A first patient has a lab for fifteen minutes, requires MA for fifteen minutes and has an appointment with physician or a nurse practitioner (MD) for fifteen minutes in sequential order starting at 08:00 and going up to 08:45. A second patient has lab for fifteen minutes, requires MA for fifteen minutes, has appointment with physician or a nurse practitioner (MD) for fifteen minutes and requires application of injection for fifteen minutes in sequential order starting at 08:00 and going up to 09:00. A third patient has appointment with physician or a nurse practitioner (MD) for fifteen minutes starting at 08:30 followed by treatment for sixty minutes going up to 09:30. fourth patient has lab for fifteen minutes starting at 8:30. A treatment profile may comprise one or more services. The optimized DOW template for this treatment profile may give many possible start times of T1, T2 and the like. This recommended set of start times in conjunction with information obtained from the EHR system described above may result in an algorithm prioritizing the time slots T1, T2 and the like. into an ordered list Ti1, Ti2 and the like. This prioritized list is then presented to an end user as part of a computer program.

In a preferred embodiment, a treatment profile may consist of one or more services. The static DOW template for the schedule date corresponding to this treatment profile may contain an ordered list of times corresponding to exact matches and approximate matches. The exact matches correspond exactly to the specific treatment profile under consideration. The approximate matches correspond in an approximate manner to the specific treatment profile under consideration.

In another embodiment, an input from the EHR system 102 such as, but not limited to, patient MRN, treatment schedule date, treatment schedule location, resources needed (such as lab chair, treatment chair and the like), resources available (such as lab chair, treatment chair, and the like), resource duration, staff schedule (such as availability of medical assistant, nurse and the like.), the number of patients assigned to each time slot and the one or more services they require, may be combined with the information provided by the optimized DOW template to derive dynamically optimized schedules for the specific treatment profile under consideration.

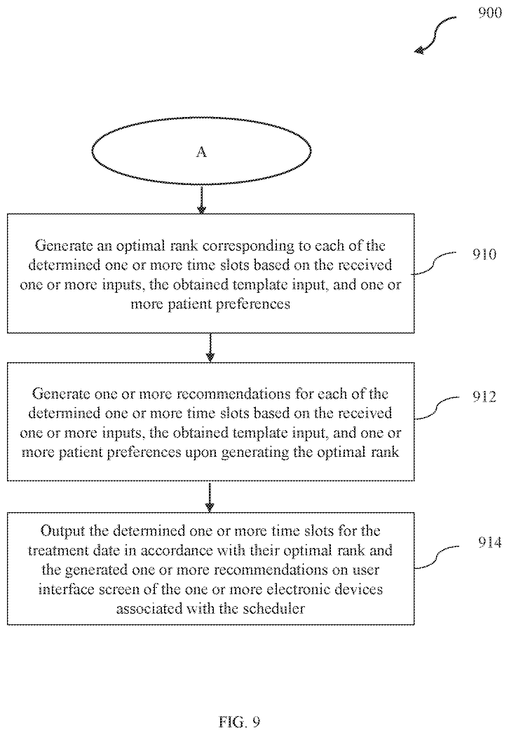

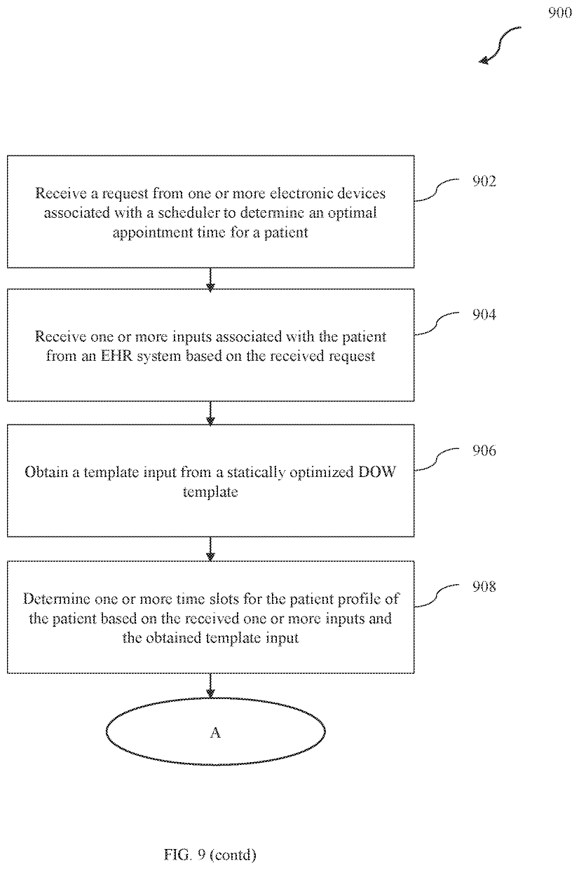

FIG. 9 is a process flow diagram illustrating an exemplary method for identifying optimal appointment times of patients, in accordance with an embodiment of the present disclosure. At step 902, a request is received from one or more electronic devices 108 associated with a scheduler to determine an optimal appointment time for a patient. In an exemplary embodiment of the present disclosure, a patient ID of the patient, a patient profile of the patient, a treatment date, and the like. In an embodiment of the present disclosure, the patient profile of the patient includes one or more medical services required by the patient. In an exemplary embodiment of the present disclosure, the patient profile corresponds to any combination of different medical services such as, but not limited to, lab tests, appointment with Medical Assistant (MA), appointment with a physician or a nurse practitioner (MD), injection, treatment, and the like. For example, the one or more medical services include injection, treatment, lab tests required to be performed on the patient, and the like. In an exemplary embodiment of the present disclosure, the one or more electronic devices 108 may include a laptop computer, desktop computer, tablet computer, smartphone, wearable device, a digital camera and the like.

At step 904, one or more inputs associated with the patient are received from the EHR system 102 based on the received request. In an exemplary embodiment of the present disclosure, the one or more inputs include a patient Medical Record Number (MRN), the treatment date, a treatment schedule location, one or more required resources, availability of the one or more required resources, a resource duration, schedule of staff, a number of patients assigned to each time slot, one or more services required by each of the patients, and the like. For example, the one or more required resources include lab chair, treatment chair, hospital sketcher, defibrillators used as part of patient treatment procedure, blood glucose meters, Magnetic Resonance Imaging (MRI), or any combination thereof. In an exemplary embodiment of the present disclosure, the staff includes availability of medical assistant, nurse, doctor, clinician, and the like.

At step 906, a clinic configuration input is Obtained from a statistically optimized Day of the Week (DOW) template. In an exemplary embodiment of the present disclosure, the statistically optimized DOW template includes forecasted patient profiles assigned to optimized time slots.

At step 908, one or more time slots for the patient profile of the patient based on the received one or more inputs and the obtained clinic configuration input. In an embodiment of the present disclosure, the one or more time slots are determined for the treatment date and a predefined clinic.

At step 910, an optimal rank corresponding to each of the determined one or more time slots is generated based on the received one or more inputs, the obtained clinic configuration input, and one or more patient preferences. In an embodiment of the present disclosure, a time slot with highest optimal rank is a most optimal time slot among the generated one or more time slots for the patient. In an embodiment of the present disclosure, a time slot with lowest optimal rank is a least optimal time slot among the generated one or more time slots for the patient. For example, the one or more patient preferences include a preferred time, a preferred staff, a preferred location of a clinic, a preferred treatment date, and the like. In generating the optimal rank corresponding to each of the determined one or more time slots based on the received one or more inputs, the obtained clinic configuration input, and the one or more patient preferences, the method 900 includes correlating the determined one or more time slots, the received one or more inputs, the obtained clinic configuration input, and the one or more patient preferences. Further, the method 900 includes generating the optimal rank corresponding to each of the determined one or more time slots based on result of correlation. For example, the computing system 104 takes into consideration different resources required by the specific treatment profile and recommends rank ordered time slots based on the EHR data and the static optimization template. The computing system 104 achieves this by combining information pertaining to the time slots assigned to various treatment profiles in the static DOW template with the specifics of the ERR data for the chosen clinic for the chosen day.

At step 912, one or more recommendations are generated for each of the determined one or more time slots based on the received one or more inputs, the obtained clinic configuration input, and one or more patient preferences upon generating the optimal rank. In an exemplary embodiment of the present disclosure, the one or more recommendations include waiting time recommendations, staff availability recommendations, resource recommendations, treatment date recommendations, and the like. For example, the one or more recommendations may be that ‘the patient may visit a clinic at the preferred time, but he may have to wait for 15 minutes.’ In another example, the one or more recommendations may be that ‘the patient may visit a clinic at the preferred time, but the one or more medical services may not be delivered by the preferred staff.’

At step 914, the determined one or more time slots are outputted for the treatment date in accordance with their optimal rank and the generated one or more recommendations on user interface screen of the one or more electronic devices 108 associated with the scheduler.

In an embodiment of the present disclosure, the method 900 includes receiving historical patient data associated with patients from the EHR system 102. In an exemplary embodiment of the present disclosure, the historical patient data include service date, unique patient identifier, a breakdown of different services required for each treatment, staff schedules, operating hours of a provider, and the like. For example, the provider may be, but not limited to a physician, group of physicians, clinic, facility that is part of a hospital or a health system, or any other person(s) or an entity that provides treatment to patients. Further, the method 900 includes generating the set of statistically optimized DOW templates for each DOW based on the received historic patient data and one or more constraints by using a statistical and combinatorial optimization analysis technique. In an embodiment of the present disclosure, the set of statistically optimized DOW templates include forecasted patient profiles for each DOW. In an exemplary embodiment of the present disclosure, the set of statistically optimized DOW templates correspond to a complete schedule for each forecasted patient profile. For example, the one or more constraints include provider schedules, staff availability, resource availability and clinic operating hours.

In a use-case scenario, a treatment profile may include one or more services. The static DOW template for this treatment profile may give many possible start times of T1, T2, . . . TN. This recommended set of start times in conjunction with information obtained from the EHR may result in prioritizing the time slots T1, T2 . . . TN into an ordered list Ti1, Ti2 . . . TiN. This prioritized list is then presented to the end user as part of a computer program. For example, a treatment profile (Tx1) may consist of 15 minutes lab, and 120 minutes treatment. This treatment profile, Tx1, may need to be scheduled for a date in the future that may be a Wednesday. Based on the static DOW template for a Wednesday, there may be three, time options when Tx1 may start; 10:00 AM (T1), 10:30 NM (T2), and 1:00 PM (T3). Information obtained from the ERR may indicate sufficient resource availability for all three start times T1, T2, and T3. Based on other considerations such as nurse availability, T2 may be considered more optimal than T3 and T3 more optimal than T1. Hence, the start times for Tx1 presented to the scheduler may be T2, T3, and T1 in that order.

In another use-case scenario, a treatment profile may include one or more services. The static day of the week template for the schedule date corresponding to this treatment profile may include an ordered list of times corresponding to “exact matches” and “approximate matches”. The “exact matches” are those that correspond exactly to the specific treatment profile under consideration whereas “approximate matches” are those that correspond in an approximate manner to the specific treatment profile under consideration. For example, a treatment profile may consist of two services, 15 minutes Lab and 60 minutes Treatment. Examples of “exact matches” for this treatment profile are: 08:00 15 minutes Lab, 08:15 60 minutes Treatment, 10:30 15 minutes Lab, 10:45 60 minutes Treatment and 15:00 15 minutes Lab, 15:15 60 minutes Treatment. Examples of “approximate matches” are: 09:30 15 minutes Lab, 09:45 90 minutes Treatment, 11:00 15 minutes Lab, 11:30 60 minutes Treatment, 14:00 30 minutes Lab, 14:15 60 minutes Treatment.

In another use-case scenario, the preferred embodiment may allow the scheduler to manually select any desired time slot or time slots for a specific treatment profile. The manual option may be used by the scheduler either when no “exact matches” or “approximate matches” are available, or when the scheduler wishes to make a selection other than the recommended optimized time slot or time slots.

The AI-based method 900 may be implemented in any suitable hardware, software, firmware, or combination thereof.

Thus, various embodiments of the present system provide a solution to facilitate identification optimal appointment times of patients. The computing system 104 comprises a novel way of identifying most optimal future appointment times for cancer patient treatments to be scheduled at cancer treatment facilities. Further, the present disclosure combines the information derived from a static optimization of forecasted patient profiles for a specific day of the week, known by those familiar with the art as a DOW template, along with dynamically changing actual schedule information obtained from the EHR system 102 for a specific future date, in an intelligent fashion to produce dynamically optimized schedules for specific treatment profiles. In an embodiment of the present disclosure, the computing system 104 discloses a novel way of identifying optimal future patient appointment times for cancer treatment, taking into consideration details such as, but not limited to, historical treatment profile data containing the service date and a breakdown of the different services needed by each treatment, staff schedules, operating hours of the provider etc. is presented. The identified optimal appointment times may be presented to an end user using a graphical user interface as part of a computer program. The computing system 104 leverages the EHR data for the chosen clinic for the chosen day and statically derived DOW optimized template data which assigns best choice time slots to forecasted patient profiles. An intelligent combination of these separately derived, highly relevant, pieces of information allows the dynamic optimized scheduler to recommend a rank ordered list of time slots for the specific patient profile under consideration.

Further, the computing system 104 creates optimized DOW template based on historic patient data to provide adaptive, and real-time patient scheduling at cancer treatment facilities. The express objective of the optimization process is to balance the load on the staff while ensuring that all resources are optimally used and the number of treatment profiles that can be accommodated is maximized. In an embodiment, the present disclosure creates customized templates to balance patient flow and resource allocation evenly during a given DOW.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments herein can comprise hardware and software elements. The embodiments that are implemented in software include but are not limited to, firmware, resident software, microcode, etc. The functions performed by various modules described herein may be implemented in other modules or combinations of other modules. For the purposes of this description, a computer-usable or computer readable medium can be any apparatus that can comprise, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

Input/output (I/O) devices (including but not limited to keyboards, displays, pointing devices, etc.) can be coupled to the system either directly or through intervening I/O. Network adapters may also be coupled to the system to enable the data processing system to become coupled to other data processing systems or remote printers or storage devices through intervening private or public networks. Modems, cable modem and Ethernet cards are just a few of the currently available types of network adapters.

A representative hardware environment for practicing the embodiments may include a hardware configuration of an information handling/computer system in accordance with the embodiments herein. The system herein comprises at least one processor or central processing unit (CPU). The CPUs are interconnected via system bus 308 to various devices such as a random-access memory (RAM), read-only memory (ROM), and an input/output (I/O) adapter. The I/O adapter can connect to peripheral devices, such as disk units and tape drives, or other program storage devices that are readable by the system. The system can read the inventive instructions on the program storage devices and follow these instructions to execute the methodology of the embodiments herein.

The system further includes a user interface adapter that connects a keyboard, mouse, speaker, microphone, and/or other user interface devices such as a touch screen device (not shown) to the bus to gather user input. Additionally, a communication adapter connects the bus to a data processing network, and a display adapter connects the bus to a display device which may be embodied as an output device such as a monitor, printer, or transmitter, for example.

A description of an embodiment with several components in communication with each other does not imply that all such components are required. On the contrary, a variety of optional components are described to illustrate the wide variety of possible embodiments of the invention. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

We claim:

1. A computing system for identifying optimal appointment times of patients, the computing system comprising:
one or more hardware processors; and
a memory coupled to the one or more hardware processors, wherein the memory comprises a plurality of modules in the form of programmable instructions executable by the one or more hardware processors, and wherein the plurality of modules comprises:
a request receiver module configured to receive a request from one or more electronic devices associated with a scheduler to determine an optimal appointment time for a patient, wherein the request comprises: a patient ID of the patient, a patient profile of the patient and a treatment date, and wherein the patient profile of the patient comprises one or more medical services required by the patient;
an input receiver module configured to receive one or more inputs associated with the patient from an Electronic Health Record (EHR) system based on the received request, wherein the one or more inputs comprise a patient Medical Record Number (MRN), the treatment date, a treatment schedule location, one or more required resources, availability of the one or more required resources, a resource duration, schedule of staff, a number of patients assigned to each time slot, and one or more services required by each of the patients;
a data obtaining module configured to obtain a clinic configuration input wherein, the clinic configuration input comprises clinic operating hours, resource count, staff count and availability constraints, first treatment start time and last treatment end time;
a schedule determination module configured to determine one or more time slots for the patient profile of the patient based on the received one or more inputs and the obtained clinic configuration input, wherein the one or more time slots are determined for the treatment date and a predefined clinic;
a rank generation module configured to generate an optimal rank corresponding to each of the determined one or more time slots based on the received one or more inputs, the obtained clinic configuration input, and one or more patient preferences, wherein a time slot with highest optimal rank is a most optimal time slot among the generated one or more time slots for the patient, and wherein a time slot with lowest optimal rank is a least optimal time slot among the generated one or more time slots for the patient;
a recommendation generation module configured to generate one or more recommendations for each of the determined one or more time slots based on the received one or more inputs, the obtained clinic configuration input, and one or more patient preferences upon generating the optimal rank; and
a data output module configured to output the determined one or more time slots for the treatment date in accordance with their optimal rank and the generated one or more recommendations on user interface screen of the one or more electronic devices associated with the scheduler.

2. The computing system of claim 1, wherein the one or more required resources comprise at least one: lab chair, treatment chair, hospital sketcher, defibrillators used as part of patient treatment procedure, blood glucose meters, and Magnetic Resonance Imaging (MRI).

3. The computing system of claim 1, wherein staff comprises availability of medical assistant, nurse, doctor, and clinician.

4. The computing system of claim 1, wherein the one or more patient preferences comprise: a preferred time, a preferred staff, a preferred location of a clinic and, a preferred treatment date.

5. The computing system of claim 1, wherein the one or more medical services comprise injection, treatment, and lab tests required to be performed on the patient.

6. The computing system of claim 1, wherein the one or more recommendations comprise waiting time recommendations, staff availability recommendations, resource recommendations and treatment date recommendations.

7. The computing system of claim 1, further comprising a template generation management module is configured to:
receive historical patient data associated with patients from the EHR system, wherein the historical patient data comprise service date, unique patient identifier, a breakdown of different services required for each treatment, staff schedules, and operating hours of a provider; and
generate a set of statistically optimized DOW templates for each DOW based on the received historic patient data and one or more constraints by using a statistical and combinatorial optimization analysis technique, wherein the set of statistically optimized DOW templates comprise forecasted patient profiles for each DOW, and wherein the set of statistically optimized DOW templates correspond to a complete schedule for each forecasted patient profile.

8. The computing system of claim 7, wherein the one or more constraints comprise provider schedules, staff availability, resource availability and clinic operating hours.

9. The computing system of claim 1, wherein in generating the optimal rank corresponding to each of the determined one or more time slots based on the received one or more inputs, the obtained clinic configuration input, and the one or more patient preferences, the rank generation module is configured to:
correlate the determined one or more time slots, the received one or more inputs, the obtained clinic configuration input, and the one or more patient preferences; and
generate the optimal rank corresponding to each of the determined one or more time slots based on result of correlation.

10. A method for identifying optimal appointment times of patients, the method comprising:
receiving, by one or more hardware processors, a request from one or more electronic devices associated with a scheduler to determine an optimal appointment time for a patient, wherein the request comprises: a patient ID of the patient, a patient profile of the patient and a treatment date, and wherein the patient profile of the patient comprises one or more medical services required by the patient;
receiving, by the one or more hardware processors, one or more inputs associated with the patient from an Electronic Health Record (EHR) system based on the received request, wherein the one or more inputs comprise a patient Medical Record Number (MRN), the treatment date, a treatment schedule location, one or more required resources, availability of the one or more required resources, a resource duration, schedule of staff, a number of patients assigned to each time slot, and one or more services required by each of the patients;
obtain a clinic configuration input, wherein, the clinic configuration input comprises clinic operating hours, resource count, staff count and availability constraints, first treatment start time and last treatment end time;
determining, by the one or more hardware processors, one or more time slots for the patient profile of the patient based on the received one or more inputs and the obtained clinic configuration input, wherein the one or more time slots are determined for the treatment date and a predefined clinic;
generating, by the one or more hardware processors, one or more recommendations for each of the determined one or more time slots based on the received one or more inputs, the obtained clinic configuration input, and one or more patient preferences upon generating the optimal rank;
generating, by the one or more hardware processors, an optimal rank corresponding to each of the determined one or more time slots based on the received one or more inputs, the obtained clinic configuration input, and one or more patient preferences, wherein a time slot with highest optimal rank is a most optimal time slot among the generated one or more time slots for the patient, and wherein a time slot with lowest optimal rank is a least optimal time slot among the generated one or more time slots for the patient;
outputting, by the one or more hardware processors, the determined one or more time slots for the treatment date in accordance with their optimal rank and the generated one or more recommendations on user interface screen of the one or more electronic devices associated with the scheduler.

11. The method of claim 10, wherein the one or more required resources comprise at least one: lab chair, treatment chair, hospital sketcher, defibrillators used as part of patient treatment procedure, blood glucose meters, and Magnetic Resonance Imaging (MRI).

12. The method of claim 10, wherein staff comprises availability of medical assistant, nurse, doctor, and clinician.

13. The method of claim 10, wherein the one or more patient preferences comprise: a preferred time, a preferred staff, a preferred location of a clinic and, a preferred treatment date.

14. The method of claim 10, wherein the one or more medical services comprise injection, treatment, and lab tests required to be performed on the patient.

15. The method of claim 10, wherein the one or more recommendations comprise waiting time recommendations, staff availability recommendations, resource recommendations and treatment date recommendations.

16. The method of claim 10, further comprising:
receiving historical patient data associated with patients from the EHR system, wherein the historical patient data comprise service date, unique patient identifier, a breakdown of different services required for each treatment, staff schedules, and operating hours of a provider; and
generating a set of statistically optimized DOW templates for each DOW based on the received historic patient data and one or more constraints by using a statistical and combinatorial optimization analysis technique, wherein the set of statistically optimized DOW templates comprise forecasted patient profiles for each DOW, and wherein the set of statistically optimized DOW templates correspond to a complete schedule for each forecasted patient profile.

17. The method of claim 10, wherein the one or more constraints comprise provider schedules, staff availability, resource availability and clinic operating hours.

18. The method of claim 15, wherein generating the optimal rank corresponding to each of the determined one or more time slots based on the received one or more inputs, the obtained clinic configuration input, and the one or more patient preferences comprises:
correlating the determined one or more time slots, the received one or more inputs, the obtained clinic configuration input, and the one or more patient preferences; and
generating the optimal rank corresponding to each of the determined one or more time slots based on result of correlation.

19. A non-transitory computer-readable storage medium having instructions stored therein that, when executed by a hardware processor, cause the processor to perform method steps comprising:
receiving a request from one or more electronic devices associated with a scheduler to determine an optimal appointment time for a patient, wherein the request comprises: a patient ID of the patient, a patient profile of the patient and a treatment date, and wherein the patient profile of the patient comprises one or more medical services required by the patient;
receiving one or more inputs associated with the patient from an Electronic Health Record (EHR) system based on the received request, wherein the one or more inputs comprise a patient Medical Record Number (MRN), the treatment date, a treatment schedule location, one or more required resources, availability of the one or more required resources, a resource duration, schedule of staff a number of patients assigned to each time slot, and one or more services required by each of the patients;
obtaining a clinic configuration input wherein, the clinic configuration input comprises clinic operating hours, resource count, staff count and availability constraints, first treatment start time and last treatment end time;
determining one or more time slots for the patient profile of the patient based on the received one or more inputs and the obtained clinic configuration input, wherein the one or more time slots are determined for the treatment date and a predefined clinic;
generating one or more recommendations for each of the determined one or more time slots based on the received one or more inputs, the obtained clinic configuration input, and one or more patient preferences upon generating the optimal rank;
generating an optimal rank corresponding to each of the determined one or more time slots based on the received one or more inputs, the obtained clinic configuration input, and one or more patient preferences, wherein a time slot with highest optimal rank is a most optimal time slot among the generated one or more time slots for the patient, and wherein a time slot with lowest optimal rank is a least optimal time slot among the generated one or more time slots for the patient; and
outputting the determined one or more time slots for the treatment date in accordance with their optimal rank and the generated one or more recommendations on user interface screen of the one or more electronic devices associated with the scheduler.

20. The non-transitory computer-readable storage medium of claim 19, wherein the one or more required resources comprise at least one: lab chair, treatment chair, hospital sketcher, defibrillators used as part of patient treatment procedure, blood glucose meters, and Magnetic Resonance Imaging (MRI).
